(19) United States
(12) Patent Application Publication
(10) Pub. No.: US 2022/0007761 A1
(43) Pub. Date: Jan. 13, 2022

(54) Anti-Static Personal Protective Equipment

(71) Applicant: Botron Co., Inc., Phoenix, AZ (US)

(72) Inventor: Keith W. Hooley, Scottsdale, AZ (US)

(73) Assignee: Botron Co., Inc., Phoenix, AZ (US)

(21) Appl. No.: 17/371,414

(22) Filed: Jul. 9, 2021

Related U.S. Application Data
(60) Provisional application No. 63/050,495, filed on Jul. 10, 2020.

(51) Int. Cl. A41D 13/11; A61F 9/02; H05F 1/02

(52) U.S. Cl. CPC A41D 13/1161; H05F 1/02; A61F 9/029

(57) ABSTRACT

Personal protective equipment includes a transparent shield, a cushion backing the shield, and a band attached to the shield to electrically couple the equipment to a user when worn. In embodiments, the band includes conductive fibers interwoven into the band to contact the user head when worn, the cushion has an electrical conductivity, and the shield has a low triboelectric effect to prevent accumulation of static charge on the shield.

### CROSS-REFERENCE TO RELATED APPLICATIONS

This application claims the benefit of U.S. Provisional Application No. 63/050,495, filed Jul. 10, 2020, which is hereby incorporated by reference.

### FIELD

The present specification relates generally to protective equipment, and more particularly to personal and electronic protective equipment.

### BACKGROUND

Static electricity is the accumulation or presence of electric charges in or on the surface of an object. The electricity is “static” because the electrical particles tend not to move, remaining on the surface until a dissipation event. Accumulation results from sliding two objects along each other, rubbing two objects against each other, or similar contact. Separating an object into two pieces also creates static electricity. Such actions generate an electrostatic voltage on a material, which may or may not dissipate quickly depending on the characteristics of the material.

The readiness with which the static electricity is dissipated depends on the conductivity of the material on which the static electricity is present. Materials which have a low resistance are considered to be conductive. Such materials transmit electricity more easily than do other materials which have a high resistance. Highly electrically-resistant materials are insulative. Typically, conductive materials have resistances below about one hundred kiloohms, while insulating materials have resistances above about one gigaohm. Between these two ranges, however, are materials which are considered to be anti-static and static-dissipative.

While static electricity itself is not well understood, the dissipation or discharge static electricity is better known. The small shocked experienced when touching a doorknob after walking across a carpeted floor is probably the most familiar example of static electricity discharge, or ESD. Lightning may perhaps be the most dramatic example of ESD. Both result from the sudden discharge of electricity that has accumulated in one medium and then transfers to the other.

The discharge of static electricity can be hazardous. For instance, when an aircraft is flying, the rapid movement of dust and precipitation creates “p-static” on the metal skin of the aircraft. P-static can affect internal electronic controls and radio operations in the airplane. Pilots hear the effects of p-static as high-pitched whining or popping over their radio systems. When p-static reaches high levels, its discharge can damage or even destroy the plane's electronic and radio components. Planes incorporate static wicks in their construction to prevent the build-up of static electricity and encourage its continuous discharge into the air to mitigate the effects of p-static while flying.

In the assembly of electronics, static electricity is also hazardous. Elements like semiconductors and transistors used in electronics are particularly sensitive to static electricity. Some components can be damaged by the discharge of accumulated charges as low as twenty volts. Most frequently, a human unfortunately causes this discharge into electronics.

A technician working on a device, assembling a device, or quality checking a device, can inadvertently introduce static electricity into a fabrication plant or manufacturing facility. As the technician walks into the fab plant, walks across the floor, and otherwise moves about in his job, his body becomes tribocharged, or charged with static electricity. This charge must eventually be discharged to ground (again, for example, by touching a doorknob). However, if the discharge occurs through the component on which the technician is working, that discharge can damage or completely ruin the component.

Currently, fab plants take precautionary measures against such inadvertent static discharge. For instance, workers wear shoes with ESD properties. These shoes prevent the accumulation of charge on the technician and readily discharge any charge that does build up. Workers also wear grounding bracelets which have a cord that the worker attaches to ground before and while working on a component. These grounding solutions have worked well in the past.

In 2020, the appearance in the United States of the novel coronavirus—the cause of the COVID-19 disease dramatically changed workspace conditions. Most businesses immediately shuttered their physical spaces and sent workers home. Essential businesses continued to remain open to provide necessary food, health supplies, and other goods and services. As time went on, more businesses opened, but companies struggled to find ways to keep employees safe while working.

Electronics manufacturing businesses often use assembly line workers who may be stationed relatively close to each other. With the emergence of COVID-19, such businesses installed plastic dividers between workers to limit the spread of exhaled air from one employee to another. However, there were and still are concerns that such dividers may not be sufficient to protect workers. As such, some businesses may use face masks or shields to further mitigate spread. While face masks and shields can reduce the transmission rates of COVID-19 and other diseases, they introduce new problems into the workplace. Conventional face shields become tribocharged, carrying as much as 15,000 volts. Improved protective equipment is needed, but such equipment must not pose an electrostatic risk to the components being built and assembled.

### SUMMARY

Personal protective equipment includes a transparent shield, a cushion backing the shield, and a band attached to the shield to electrically couple the equipment to a user when worn. In embodiments, the band includes conductive fibers interwoven into the band to contact the user head when worn, the cushion has an electrical conductivity, and the shield has a low triboelectric effect to prevent accumulation of static charge on the shield.

The above provides the reader with a very brief summary of some embodiments described below. Simplifications and omissions are made, and the summary is not intended to limit or define in any way the disclosure. Rather, this brief summary merely introduces the reader to some aspects of some embodiments in preparation for the detailed description that follows.

### BRIEF DESCRIPTION OF THE DRAWINGS

Referring to the drawings:

FIG. 1 is a perspective view of personal protective equipment;

FIG. 2 is an exploded perspective view of the personal protective equipment of FIG. 1;

FIG. 3 is a front elevation view of the personal protective equipment of FIG. 1; and

FIG. 4 is a top plan view of the personal protective equipment of FIG. 1.

### DETAILED DESCRIPTION

Reference now is made to the drawings, in which the same reference characters are used throughout the different figures to designate the same elements. Briefly, the embodiments presented herein are preferred exemplary embodiments and are not intended to limit the scope, applicability, or configuration of all possible embodiments, but rather to provide an enabling description for all possible embodiments within the scope and spirit of the specification. Description of these preferred embodiments is generally made with the use of verbs such as “is” and “are” rather than “may,” “could,” “includes,” “comprises,” and the like, because the description is made with reference to the drawings presented. One having ordinary skill in the art will understand that changes may be made in the structure, arrangement, number, and function of elements and features without departing from the scope and spirit of the specification. Further, the description may omit certain information which is readily known to one having ordinary skill in the art to prevent crowding the description with detail which is not necessary for enablement. Indeed, the diction used herein is meant to be readable and informational rather than to delineate and limit the specification; therefore, the scope and spirit of the specification should not be limited by the following description and its language choices.

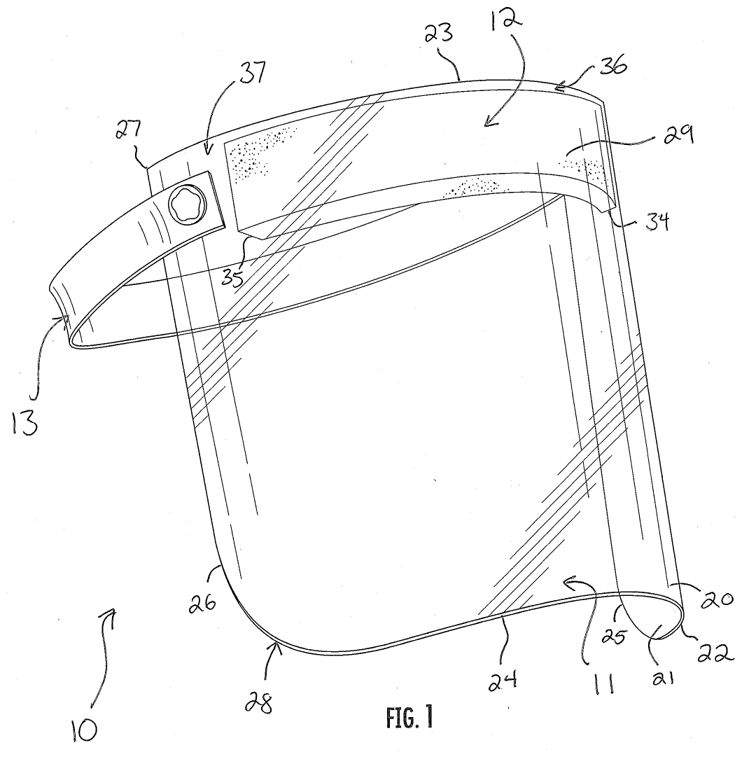

FIG. 1 illustrates an anti-static face shield or personal protective equipment (“PPE”) 10. The term “PPE” is generally used in this description to identify the anti-static face shield. The PPE 10 reduces airborne transmission of viruses, pathogens, and other harmful elements. The PPE 10 effectively prevents droplets originating in the user's lungs from being exhaled out of the mouth and nose and into the surrounding air. Further, the PPE 10 is safe to use in ESD-sensitive areas; it mitigates the risk of accidental ESD damaging electronic components on which the user may be working. The PPE 10 includes a transparent shield 11, a cushion 12 backing the shield 11, and a band 13 attached to the shield 11 for placing around the user's head while the PPE 10 is worn.

The shield 11 is transparent so that, when the PPE 10 is worn, the user can maintain vision without obstruction. The shield 11 is a thin sheet of material permanently bent into a concave shape approximately corresponding to the shape of a human head.

The shield 11 has a front surface 20, an opposed rear surface 21, and a perimeter edge 22. The perimeter edge 22 extends continuously and entirely around the shield 11 and is formed by the combination of a top edge 23, an opposed bottom edge 24, and opposed left and right side edges 25 and 26. The top edge 23 extends linearly, meeting each of the opposed left and right side edges 25 and 26 at defined corners 27 of approximately ninety degrees in angle. Opposite the top edge 23, the opposed left and right side edges 25 and 26 curve at opposed large rounded corners 28 arcuately downward and then inward to the bottom edge 24. The bottom edge 24 is linear between these opposed corners 28.

Because the shield 11 is concave around the user's face, the top and bottom edges 23 and 24 curve backward in correspondence to the human head shape. The shield 11 has an apex 29 which is the most forward portion of the shield 11 projecting away from the user. When worn, the rear surface 21 is presented toward the user and the front surface 20 is away therefrom; the apex 29 is a line extending vertically down the shield 11 approximately equidistant between the opposed left and right side edges 25 and 26. From the apex 29, the shield 11 bends rearwardly to curve around the user's face, terminating at the left and right side edges 25 and 26, which are preferably but not necessarily linear and approximately vertical.

The shield 11 is extremely thin. It is preferably but not necessarily flexible. It is constructed from an anti-static material, such as a polycarbonate, polyethylene terephthalate glycol, acrylic, or like material which has a low triboelectric effect such that it prevents the accumulation of static charge. The shield 11 is thus ESD safe. This serves not only to prevent static electric attraction of items like hair, plastic film, papers, and the like which may obscure the user's vision, but to reduce the likelihood of ESD. Because the shield 11 can hold little to no electrostatic charge, the discharge of that charge—and the potential for damage from such discharge—is lessened. As such, the shield 11 material is anti-static.

In other embodiments, the shield 11 is treated with an anti-static coating. The anti-static coating is not shown in the drawings, as it is a clear, invisible coating applied to the front surface 20, rear surface 21, and perimeter edge 22 to encase the shield 11 with a protective anti-static layer. The anti-static coating may be applied to shield 11 materials which are anti-static to improve the existing anti-static characteristics, or may be applied and reapplied to shield 11 materials with less favorable anti-static qualities to reach the desired anti-static level. Testing has shown that, in operation, the shield 11 has a low triboelectric effect, with a surface resistance of between approximately one megaohm and approximately one hundred gigaohms, and accumulating preferably no more than approximately 250 volts which is then dissipated to zero or near zero volts almost immediately and within one to two seconds. In contrast, conventionally-constructed shields accumulate charges of around 2,000 to 15,000 volts without readily dissipating that charge.

The shield 11 is also resistant to fogging: in some embodiments, the shield 11 is treated with an antifog coating, while in other embodiments, the shield 11 is constructed from an intrinsically anti-fog material.

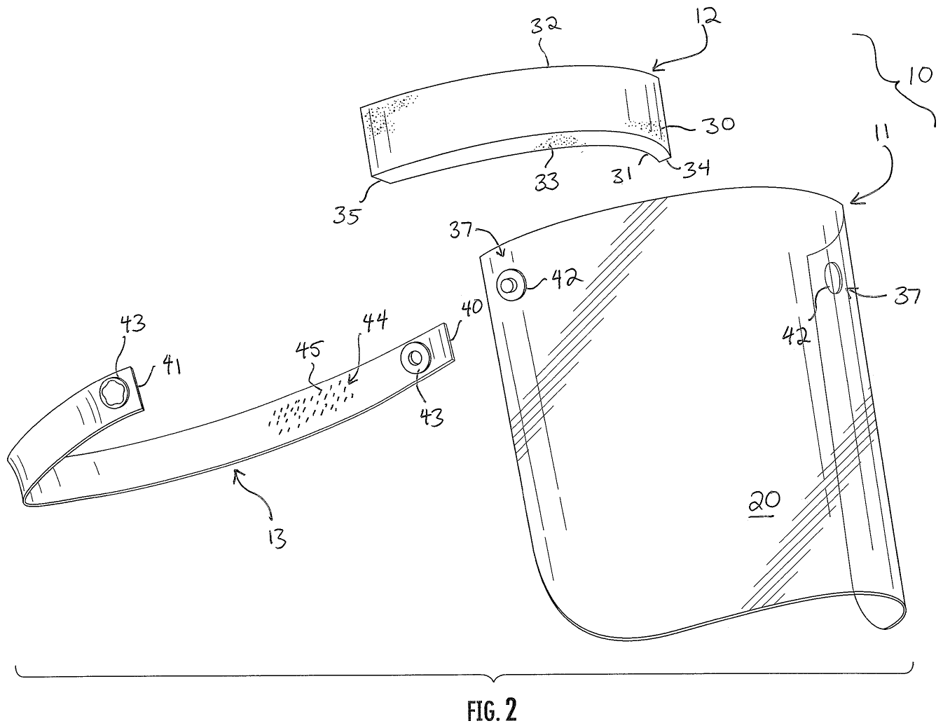

The cushion 12 is a comfort pad affixed to the shield 11. It provides padding against the user's head and provides a grounding or dissipation path for the PPE 10. Referring primarily to FIG. 2, the cushion 12 is preferably, but not necessarily, an elongate rectangular prism. The cushion 12 has a front surface 30 which is adhered to the rear surface of the shield 11, and an opposed rear surface 31. The cushion 12 has a top edge 32 and an opposed bottom edge 33. The front and rear surfaces 30 and 31 are broad and flat.

The cushion 12 extends between opposed left and right sides 34 and 35. As shown in FIG. 1, the cushion 12 is disposed against the rear surface 21 of the shield 11 such that the top edge 32 of the cushion 12 is proximate the top edge 23 of the shield 11. In some embodiments, the top edge 32 is above the top edge 23, while in other embodiments it is level with or below the top edge 23. In the embodiment shown here, the top edge 32 of the cushion 12 is just below the top edge 23, defining a thin and long gap 36 between those edges 32 and 23.

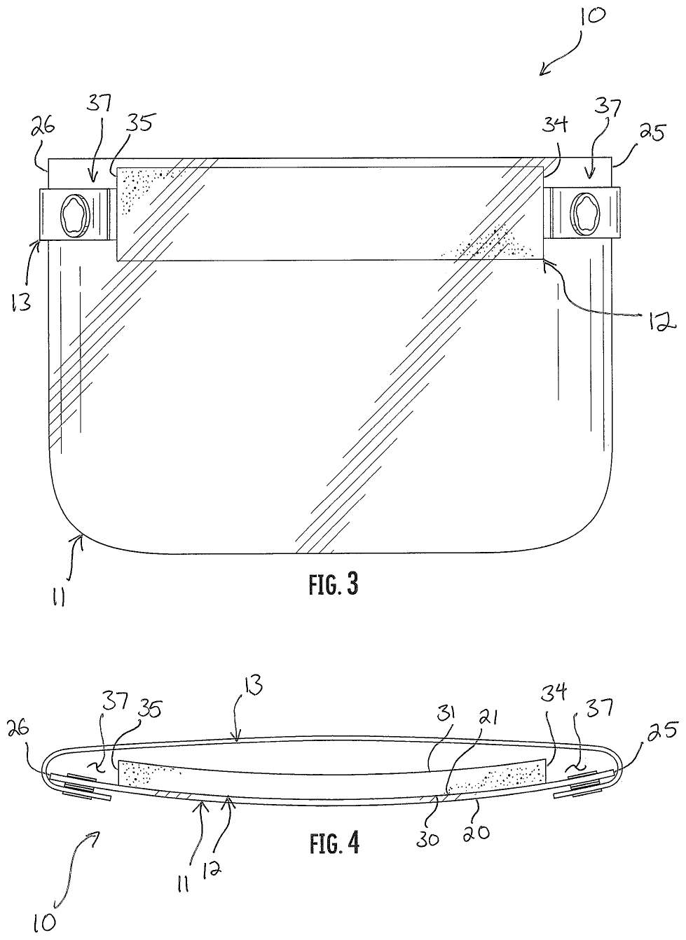

The cushion 12 preferably, but not necessarily, extends just short of the entirety between the left and right side edges 25 and 26 of the shield 11. Instead of extending laterally across the entire rear surface 21 of the shield 11, the left and right sides 34 and 35 of the cushion 12 stop short of, or are inboard of, the left and right side edges 25 and 26, respectively. As such, gaps 37 are formed between the left side 34 and the left side edge 25 and between the right side 35 and the right side edge 26. FIG. 1 shows the gap 37 between the left side 34 and the left side edge 25; the opposing gap 37 is hidden in this view. However, FIGS. 3 and 4, a front elevation view and a top plan view, respectively, show both gaps 37 clearly.

The cushion 12 is preferably constructed from an anti-static polyethylene foam chemically doped or infused with an anti-static agent or surfactant. The cushion 12 is a closed-cell, chemically cross-linked anti-static, ESD-safe foam with a low triboelectric effect. The cushion 12 is preferably, but not necessarily, between one-half and two inches in thickness between its front and rear surfaces 30 and 31, though other dimensions may be suitable as well. Preferably, the foam of the cushion 12 has a surface resistance of less than approximately one hundred gigaohms, though values outside this range may be suitable in some embodiments of the PPE 10 as well, and this range is not meant to limit the scope of the disclosure. Moreover, when doped with the anti-static agent, the cushion 12 has a surface resistance between approximately one hundred kilohms and approximately one hundred gigaohms, and preferably between approximately one megaohm and approximately one gigaohm. This cushion 12 generates a very low electrostatic charge (especially when compared to conventional foam cushions) when rubbed against itself or another material but does dissipate or pass electrostatic charges readily. As such, when the cushion 12 is worn against the user's head and the user is grounded, the cushion 12 provides a grounding path through the user for ESD. This grounding path provides for more immediate dissipation of electrical charge, with potential accumulation of below 250 volts and dissipation to zero or near zero volts within one to two seconds.

The band 13 is also affixed to the shield 11. Referring primarily to FIG. 2, the band 13 holds the PPE 10 to the user when the user wears the PPE 10 against his head. Additionally, the band 13 has anti-static properties. The band 13 is a thin piece of elastic material having two opposed free ends 40 and 41. The free ends 40 and 41 of the band 13 are affixed to the shield 11, preferably at the gaps 37. As shown in FIG. 2, snap closure bases 42 are mounted through the shield 11 at the gaps 37. Complemental snap closure caps 43 are carried at the free ends 40 and 41 of the band 13. The bases 42 each include posts projecting outwardly away from the front surface 20 of the shield 11, and the caps 43 have sockets which receive the posts. As such, the band 13 may be attached to the shield 11 against the front surface 20 thereof, on the opposite side of the shield 11 from the cushion 12. The band 13 itself is a single universal size.

In other embodiments, the free ends 40 and 41 are secured to the shield 11 at the gaps 37 with buckles or clips that allow the effective length of the band 13 to be adjusted by pulling the free ends 40 and 41 through the clips. Such clips are preferably glued or riveted through the shield 11. In yet other embodiments, the shield 11 has slots though which the band 13 is looped. And in yet still other embodiments, the band 13 is connected to the shield 11 with a conductive hook-and-loop engagement.

The band 13 is preferably constructed from a woven blend of cotton or nylon and elastic materials having a low triboelectric effect. Conductive fibers 44, such as short aluminum fibers, are interwoven into the band 13. In some embodiments, silver-coated fibers are used in place of the aluminum fibers. These conductive fibers 44 have a plurality of short, small free ends 45 that jut or project out of the band 13. When the band 13 is worn, these free ends 45 contact the user's skin and create a continuous electric path between the band 13 and the user's skin. As such, the band 13 establishes a grounding path to ground through the user. Some of the fibers 44 and their free ends 45 are shown in FIG. 2; the reader will understand that the fibers 44 are interwoven throughout the band 13 and that the free ends 45 project inward from the surface of the band 13 toward the user's head. If the user wears the PPE 10 such that the band 13 is placed over hair only, then the band 13 grounds through the shield 11, the cushion 12, and finally the user.

In operation, a user wears the PPE 10 by holding the shield 11 in front of his face, placing the cushion 12 against the skin of his forehead, and pulling the band 13 back and around his head. The elastic of the band 13 constricts the band 13 around the head and maintains the position of the PPE 10 on the user's head. As the user moves, any charges that accumulate are quickly dissipated off of the PPE 10, either through a continuity grounding path to ground through the shield 11, the cushion 12, and the user, or through another continuity path to ground through the shield 11, the conductive fibers 44 of the band 13, and the user. In other words, the grounding path includes the shield 11, the cushion 12, the band 13, and the user. As such, the PPE 10 effectively prevents the accumulation of electrostatic charges which might damage electronics. Initial tests have shown that, when worn in this fashion, the shield 11 accumulates no more than a 250 volt potential but that this potential discharges immediately, within one to two seconds of accumulation.

A preferred embodiment is fully and clearly described above so as to enable one having skill in the art to understand, make, and use the same. Those skilled in the art will recognize that modifications may be made to the description above without departing from the spirit of the specification, and that some embodiments include only those elements and features described, or a subset thereof. To the extent that modifications do not depart from the spirit of the specification, they are intended to be included within the scope thereof.

What is claimed is:

1. A personal protective equipment comprising:
a transparent shield;
a cushion backing the shield; and
a band attached to the shield configured to electrically couple the equipment to a user when worn on a user head;
wherein the band includes conductive fibers interwoven into the band to contact the user head when worn.

2. The personal protective equipment of claim 1, wherein the conductive fibers have free ends which project out from the band to contact the user head when the equipment is worn.

3. The personal protective equipment of claim 1, wherein the cushion has an electrical resistance between one megaohm and one gigaohm.

4. The personal protective equipment of claim 1, wherein the cushion is doped with an anti-static agent.

5. The personal protective equipment of claim 1, wherein the shield has a low triboelectric effect to prevent accumulation of static charge on the shield.

6. The personal protective equipment of claim 1, wherein the shield is constructed from an anti-static material.

7. The personal protective equipment of claim 1, wherein the shield is coated with an anti-static coating.

8. A personal protective equipment comprising:
a transparent shield;
a cushion backing the shield; and
a band attached to the shield configured to electrically couple the equipment to a user when worn on a user head;
wherein the cushion has an electrical resistance between one megaohm and one gigaohm.

9. The personal protective equipment of claim 8, wherein the cushion is doped with an anti-static agent.

10. The personal protective equipment of claim 8, wherein the shield has a low triboelectric effect to prevent accumulation of static charge on the shield.

11. The personal protective equipment of claim 8, wherein the shield is constructed from an anti-static material.

12. The personal protective equipment of claim 8, wherein the shield is coated with an anti-static coating.

13. The personal protective equipment of claim 8, wherein the band includes conductive fibers interwoven into the band to contact the user head when the equipment is worn.

14. The personal protective equipment of claim 13, wherein the conductive fibers include free ends which project out from the band to contact the user head when the equipment is worn.

15. A personal protective equipment comprising:
a transparent shield;
a cushion backing the shield; and
a band attached to the shield configured to electrically couple the equipment to a user when worn on a user head;
wherein the shield has a low triboelectric effect to prevent accumulation of static charge on the shield.

16. The personal protective equipment of claim 15, wherein the shield is constructed from an anti-static material.

17. The personal protective equipment of claim 15, wherein the shield is coated with an anti-static coating.

18. The personal protective equipment of claim 15, wherein the band includes conductive fibers interwoven into the band to contact the user head when the equipment is worn.

19. The personal protective equipment of claim 18, wherein the conductive fibers include free ends which project out from the band to contact the user head when the equipment is worn.

20. The personal protective equipment of claim 15, wherein the cushion has an electrical resistance between one megaohm and one gigaohm.

21. The personal protective equipment of claim 20, wherein the cushion is doped with an anti-static agent.
